(19) United States
(12) Patent Application Publication
(10) Pub. No.: US 2021/0090740 A1
(43) Pub. Date: Mar. 25, 2021

(54) STATE ESTIMATION APPARATUS, STATE ESTIMATION METHOD, AND NON-TRANSITORY RECORDING MEDIUM

(71) Applicant: CASIO COMPUTER CO., LTD., Tokyo (JP)

(72) Inventors: Kouichi NAKAGOME, Tokorozawa-shi (JP); Yasushi MAENO, Tokyo (JP); Takashi YAMAYA, Tokyo (JP); Mitsuyasu NAKAJIMA, Tokyo (JP); Kazuhisa MATSUNAGA, Tokyo (JP)

(73) Assignee: CASIO COMPUTER CO., LTD., Tokyo (JP)

(21) Appl. No.: 17/029,839

(22) Filed: Sep. 23, 2020

(30) Foreign Application Priority Data
Sep. 24, 2019 (JP) 2019-172841

(51) Int. Cl. G16H 50/30; A61B 5/0205; A61B 5/00; G16H 40/67

(52) U.S. Cl. CPC G16H 50/30; A61B 5/0205; A61B 5/024; G16H 40/67; A61B 5/7278

(57) ABSTRACT

A state estimation apparatus includes: at least one processor; and a memory configured to store a program executable by the at least one processor; wherein the at least one processor is configured to: acquire a biological signal of a subject, in a certain period in which the biological signal is being acquired, set as a plurality of extraction time windows a plurality of time windows having mutually different time lengths, extract a feature value of the biological signal in each of the plurality time windows, and estimate a state of the subject based on the extracted feature value.

### CROSS-REFERENCE TO RELATED APPLICATION

This application claims the benefit of Japanese Patent Application No. 2019-172841, filed on Sep. 24, 2019, the entire disclosure of which is incorporated by reference herein.

### FIELD

The present disclosure relates to a state estimation apparatus, a state estimation method, and a non-transitory recording medium.

### BACKGROUND

Apparatuses for predicting the state of a subject are known in the art. An example of such an apparatus is disclosed in Japanese Unexamined Patent Application No. 2015-217130. This conventional apparatus is configured to estimate the sleep state of a person. This apparatus includes a sensor that consists of a piezoelectric element and that is worn on the chest of the person. Additionally, in this conventional apparatus, a certain determination unit time (hereinafter referred to as “epoch”) in respiratory waveform data of the person detected by the sensor is set. Moreover, for each epoch, a plurality of feature values such as a peak-to-peak difference of the respiratory waveform data is extracted, and the extracted feature values are used to estimate which of three stages, namely “awake”, “light sleep”, and “deep sleep”, the sleep state of the person is in.

### SUMMARY

A state estimation apparatus according to the present disclosure includes:

at least one processor; and

a memory configured to store a program executable by the at least one processor;

wherein

the at least one processor is configured to:
acquire a biological signal of a subject,
in a certain period in which the biological signal is being acquired, set as a plurality of extraction time windows a plurality of time windows having mutually different time lengths,
extract a feature value of the biological signal in each of the plurality time windows, and
estimate a state of the subject based on the extracted feature value.

### BRIEF DESCRIPTION OF THE DRAWINGS

A more complete understanding of this application can be obtained when the following detailed description is considered in conjunction with the following drawings, in which:

FIG. 1 is a drawing illustrating the mechanical configuration of a state estimation apparatus according to an embodiment of the present disclosure;

FIG. 2 is a drawing illustrating the functional configuration of the state estimation apparatus according to an embodiment of the present disclosure;

FIG. 3 is a drawing illustrating a detection waveform of a pulse wave sensor according to an embodiment of the present disclosure;

FIG. 4 is a flowchart illustrating sleep state estimation processing according to an embodiment of the present disclosure;

FIG. 5 is a flowchart illustrating biological signal acquisition processing according to an embodiment of the present disclosure;

FIG. 6 is a drawing illustrating an example of an estimated heartbeat interval according to an embodiment of the present disclosure;

FIG. 7 is a flowchart illustrating feature value extraction processing according to an embodiment of the present disclosure;

FIG. 8 is a drawing illustrating an example of re-sampling of the heartbeat interval according to an embodiment of the present disclosure;

FIG. 9 is a flowchart illustrating frequency-based feature value extraction processing according to an embodiment of the present disclosure;

FIG. 10 is a drawing explaining extraction time windows used to extract a data string according to an embodiment of the present disclosure;

FIG. 11 is a flowchart illustrating respiration-based feature value extraction processing according to an embodiment of the present disclosure;

FIG. 12 is a flowchart illustrating time-based feature value extraction processing according to an embodiment of the present disclosure; and

FIG. 13 is a drawing illustrating an estimator according to an embodiment of the present disclosure.

### DETAILED DESCRIPTION

Hereinafter, a feature value extraction apparatus, a state estimation apparatus, a feature value extraction method, a state estimation method, and a non-transitory recording medium according to embodiments of the present disclosure are described while referencing the drawings.

### Embodiment 1

In the present embodiment, an example is described in which a sleep state of a human, which is a subject, is predicted as the state of the subject.

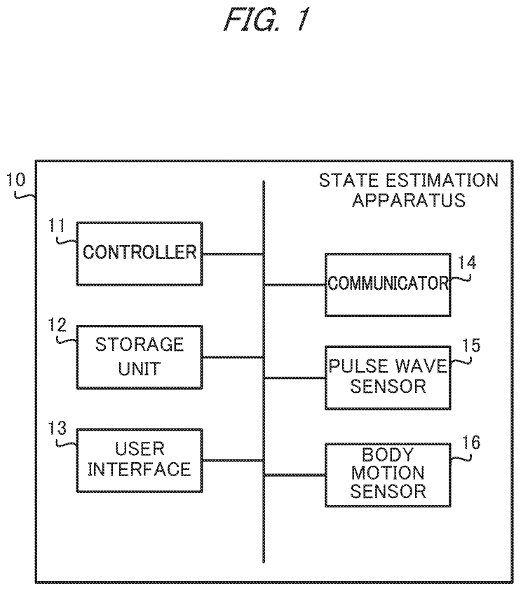

As illustrated in FIG. 1, a state estimation apparatus 10 according to the present embodiment physically includes a controller 11, a storage unit 12, a user interface 13, a communicator 14, a pulse wave sensor 15, and a body motion sensor 16.

The controller 11 includes at least one central processing unit (CPU), a read-only memory (ROM), and a random access memory (RAM). In one example, the CPU is a microprocessor or the like and is a central processing unit that executes a variety of processing and computation. In the controller 11, the CPU is connected, via a system bus, to each component of the state estimation apparatus 10. Additionally, the CPU functions as a control device that reads a control program stored in the ROM and controls the operations of the entire state estimation apparatus 10 while using the RAM as working memory.

The storage unit 12 is nonvolatile memory such as flash memory or a hard disk. The storage unit 12 stores programs and data used by the controller 11 to perform various processes. In one example, the storage unit 12 stores biological data that serves as teacher data, and tables in which various settings for biological state estimation are set. Moreover, the storage unit 12 stores data generated or acquired as a result of the controller 11 performing the various processes.

The user interface 13 includes input device such as input keys, buttons, switches, touch pads, and touch panels; a display device such as a liquid crystal panel and a light emitting diode (LED); a sound outputter such as a speaker or a buzzer; and a vibration device such as a vibrator. The user interface 13 receives various operation commands from the user via the input device, and sends the received operation commands to the controller 11. Moreover, the user interface 13 acquires various information from the controller 11 and displays images that represent the acquired information on the display device.

The communicator 14 includes an interface that enables the state estimation apparatus 10 to communicate with external devices. Examples of the external devices include personal computers, tablet terminals, smartphones, and other terminal devices. The communicator 14 communicates with the external devices via, for example, a universal serial bus (USB), a wireless local area network (LAN) such as wireless fidelity (Wi-Fi), Bluetooth (registered trademark), or the like. Under the control of the controller 11, the communicator 14 acquires, via such wired or wireless communication, various types of data, including teacher data, from the external devices. Note that the data described above may be stored in advance in the storage unit 12.

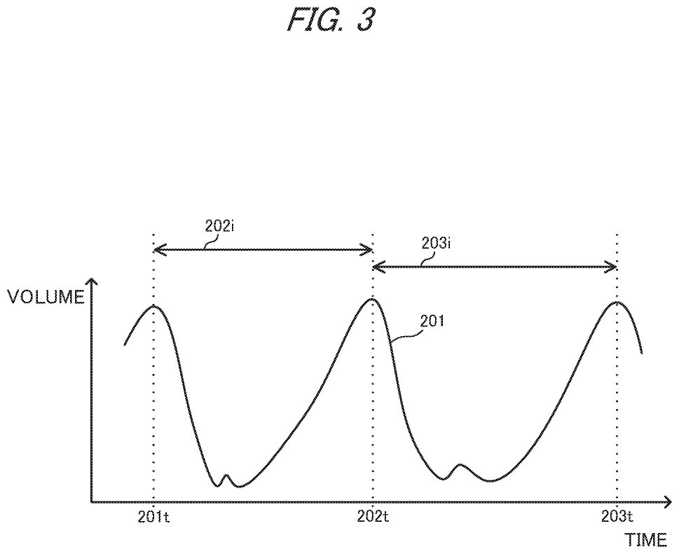

The pulse wave sensor 15 detects a pulse wave, which is one of the biological signals of the subject. In one example, the pulse wave sensor 15 is worn on an earlobe, an arm, a finger, or the like of the subject. The pulse wave sensor 15 detects changes in the amount of light absorbed by oxyhemoglobin in the blood by irradiating light from the surface of the skin of the subject and measuring the reflected light or the transmitted light thereof. These changes in the amount of absorbed light correspond to changes in blood vessel volume. As such, the pulse wave sensor 15 obtains a pulse wave 201 that captures changes in blood vessel volume as a waveform, as illustrated in FIG. 3.

Moreover, since it is thought that changes in blood vessel volume are synchronized with the beating of the heart, it is possible to estimate the timing (point in time) when the amplitude of the pulse wave peaks as the timing when a beat of the heart occurs (hereinafter referred to as “beat timing”). Additionally, it is possible to estimate, as a heartbeat interval (R-R interval, hereinafter referred to as “RRI”), a time interval between two adjacent peaks of the pulse wave on the time axis. Accordingly, the controller 11 can estimate the RRI based on the pulse wave detected by the pulse wave sensor 15. The beat timing can, for example, be estimated using a timer (or a clock) that a CPU has, or a sampling frequency of a pulse wave, but may also be predicted using other appropriate methods. In FIG. 3, the timings at which the pulse wave 201 peaks correspond to beat timings 201t, 202t, 203t, and the intervals between the peaks of the pulse wave 201 correspond to RRI 202i and RRI 203i. The present embodiment will be described using an example in which the pulse wave (heartbeat) is detected using LED light. It goes without saying that the electrical signals of the heart may be directly detected.

The body motion sensor 16 is implemented as an acceleration sensor that detects, as the motion of the body of the subject, acceleration in the directions of three axes, namely the X, Y, and Z axes, which are orthogonal to each other. In one example, the body motion sensor 16 is attached to an earlobe of the subject. A gyrosensor, a piezoelectric sensor, or the like may be used as the body motion sensor 16.

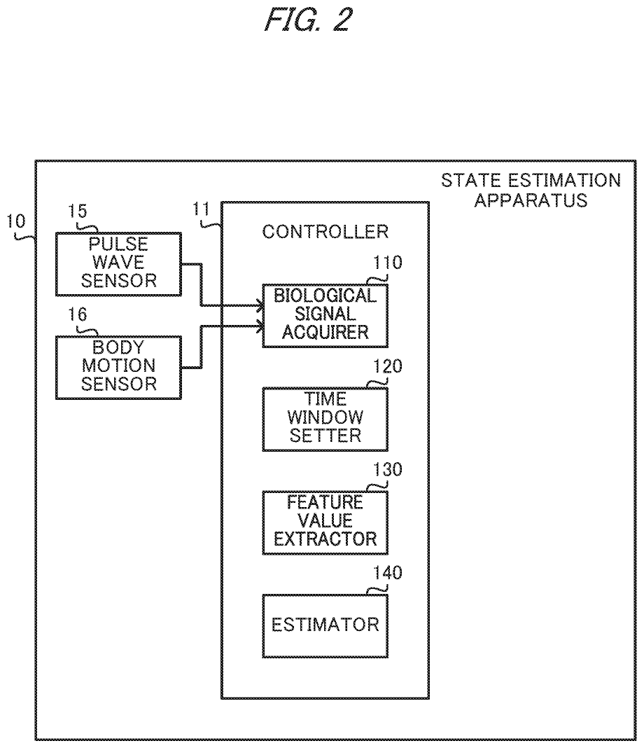

Next, the functional configuration of the controller 11 of the state estimation apparatus 10 is described while referencing FIG. 2. As illustrated in FIG. 2, the state estimation apparatus 10 functionally includes a biological signal acquirer 110 that is an acquisition device for acquiring a biological signal of the subject, a time window setter 120 that is a setting device that sets extraction time windows for extracting feature values from the biological signal acquired by the biological signal acquirer 110, a feature value extractor 130 that is an extraction device that extracts the feature values from the biological signal in the ranges of the extraction time windows set by the time window setter 120, and an estimator 140 that is a estimation device that estimates the sleep state based on the extracted feature values. In the controller 11, the CPU reads the program stored in the ROM out to the RAM and executes that program, thereby functioning as the various components described above. Note that, the functional components of the state estimation apparatus 10, with the exception of the estimator 140, form the feature value extraction apparatus according to Embodiment 1.

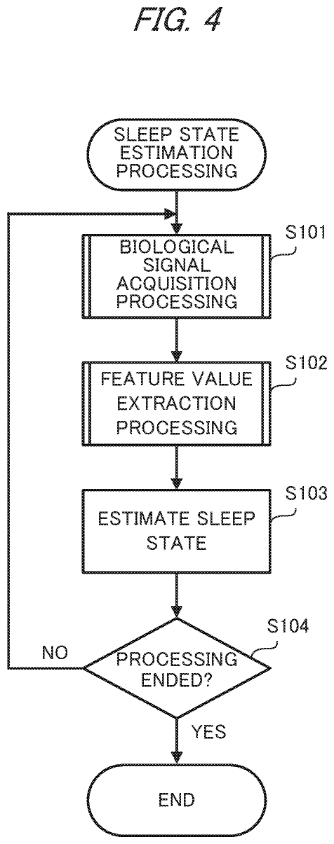

Next, while referencing FIG. 4, the various functions of the state estimation apparatus 10 will be described together with the flow of processing of sleep state estimation processing. The sleep state estimation processing starts when the state estimation apparatus 10 is started up. Alternatively, the sleep state estimation processing may start when the state estimation apparatus 10 receives an instruction from a user via an input device. First, the biological signal acquirer 110 of the state estimation apparatus 10 executes biological signal acquisition processing (step S101).

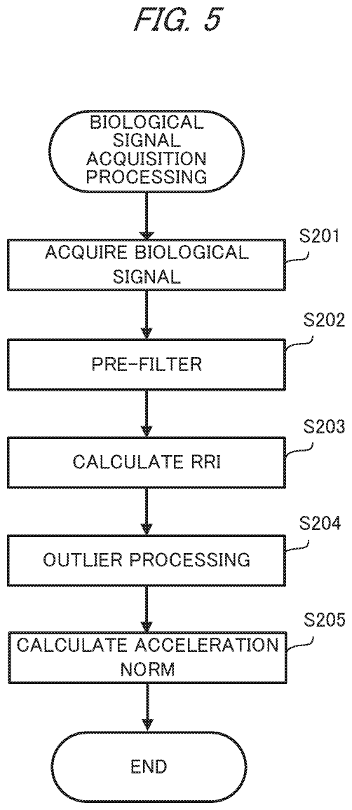

FIG. 5 illustrates a flowchart of the biological signal acquisition processing. First, the biological signal acquirer 110 acquires a pulse wave signal from the pulse wave sensor 15 and a body motion signal from the body motion sensor 16 (step S201). The pulse wave signal acquired from the pulse wave sensor 15 is sampled at a certain frequency (for example, 250 Hz), and the sampled pulse wave signal is filtered by a pre-filter constituted by a band-pass filter to remove low frequency components and high frequency noise (step S202). Then, the RRIs are calculated (estimated) from the filtered pulse wave signal (step S203).

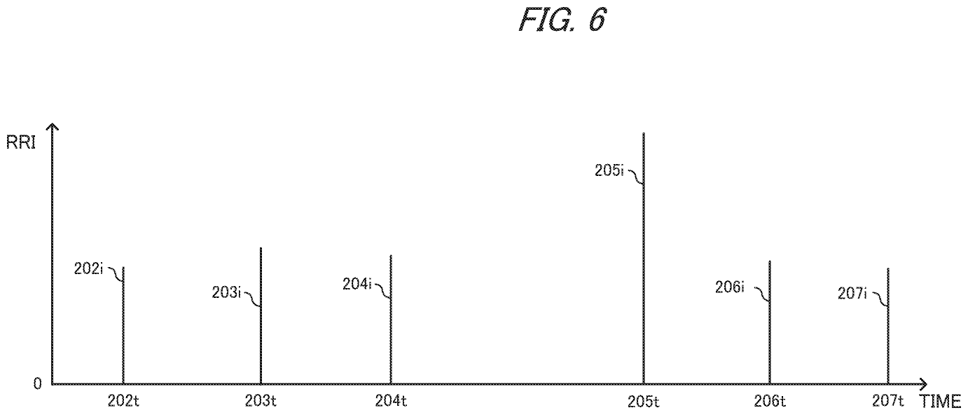

There are various methods for calculating the RRIs. Examples thereof include methods using correlation functions, methods using Fast Fourier Transform (FFT), methods for calculating based on detecting the maximum amplitude value, and the like. For example, in a case in which the RRIs are calculated based on the detection of the maximum amplitude value, the maximum amplitude value is detected from the pulse wave signal and compared with a plurality of maximum amplitude values detected within a certain amount of time before and after the timing at which the maximum amplitude value is detected. As a result of the comparison, the timing at which the greatest maximum amplitude value is detected is estimated as the beat timing. This type of comparison is carried out for each maximum amplitude value of the pulse wave signal, and the time interval between two adjacent beat timings on the time axis is calculated as an RRI. A graph such as that illustrated in FIG. 6 is obtained by plotting the calculated RRIs with time on the horizontal axis and the RRI on the vertical axis. In FIG. 6, the intervals between the two adjacent beat timings (202t and the like) corresponds to the RRI. In this case, an example is illustrated of a case in which a beat that is expected could not be calculated between beat timing 204t and beat timing 205t.

When calculating the RRIs, outlier processing is carried out for removing outliers (abnormal values) of the RRIs (step S204). In one example, the outlier processing is carried out as follows. The total range of the calculated RRIs is sectioned into set intervals (for example, 2 seconds), and the standard deviation of each of the divided sections is calculated. If the calculated standard deviation is less than a certain threshold, the RRI of that section is considered to be an outlier and is removed. Additionally, if the RRI is not in a certain range that is typically expected, the RRI of that section is considered to be an outlier and is removed since the resting RRI is not satisfied. Furthermore, if the difference between adjacent RRIs exceeds a certain value, both of the adjacent RRIs are considered to be outliers and removed since rapid variation in the RRIs is unlikely. In FIG. 6, the RRI 205i is considered to be an outlier and is removed.

Next, the biological signal acquirer 110 samples, at a certain sampling rate (for example, 30 Hz) the body motion signal acquired in step S201 or, in order words, samples the accelerations in the three axial directions of the X, Y, and Z axes; and further samples at a rate (for example 2 Hz) lower than the sampling rate to acquire time series data of acceleration. The biological signal acquirer 110 calculates the magnitude (norm) of a composite vector of the acceleration in the X, Y, and Z axial directions from the acquired acceleration data (step S205). While not illustrated in the drawings, the calculated norm of the composite vector is filtered using a band-pass filter to remove noise. In this case, the pass band of the band-pass filter is set to 0.05 Hz to 0.25 Hz, for example.

In FIG. 4, when the biological signal acquirer 110 ends the biological signal acquisition processing, the feature value extractor 130 executes feature value extraction processing for extracting feature values from the RRIs and the acceleration norms calculated by the biological signal acquirer 110 (step S102). The feature values are extracted in accordance with the extraction time windows set by the time window setter 120.

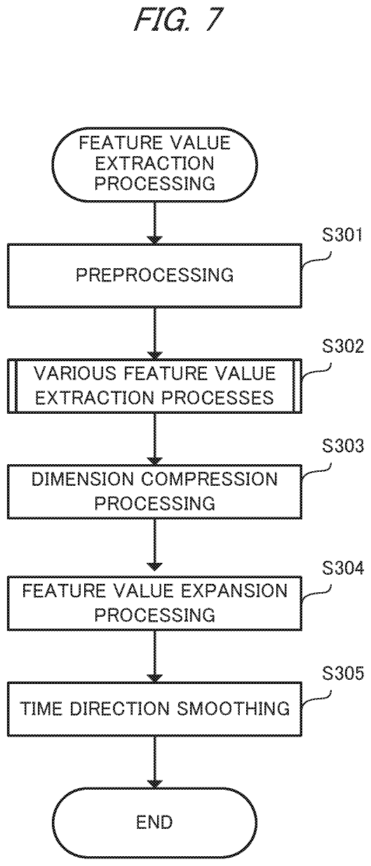

The feature value extractor 130 extracts, as feature values, frequency-based feature values that are feature values based on the frequency of the RRIs, respiration-based feature values that are feature values based on a respiratory signal extracted from the RRIs, and time-based feature values that are feature values based on the time of the RRIs and the time of the acceleration norms. Since the RRI is based on the pulse wave signal of the subject, these feature values can be considered to be feature values of the pulse wave signal. Moreover, since the acceleration norm is based on the body motion signal of the subject, the time-based feature values can be considered to be feature values of the body motion signal. The feature value extraction processing executed in step S102 will be described in order while referencing the flowchart of FIG. 7.

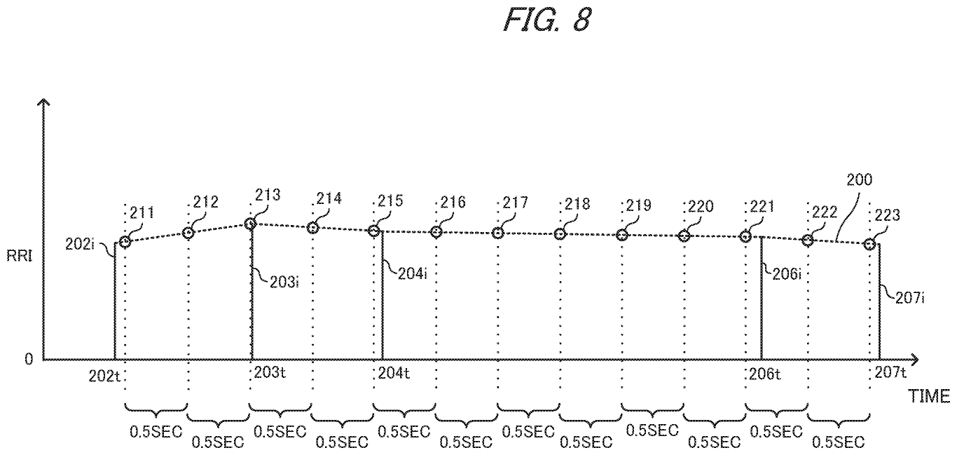

When the RRIs are calculated, as preprocessing for the feature value extraction, the feature value extractor 130 executes processing for generating equal-interval RRIs by aligning, at equal intervals, the irregularities of the sampling intervals caused by fluctuations of the RRIs (step S301). Making the sampling intervals equal intervals enables the FFT processing executed to extract the feature values. For example, as illustrated in FIG. 8, the dotted line 200 is obtained by linearly interpolating (spline interpolation and the like is also possible) the RRIs after the outlier removal, and the values on the interpolated dotted line 200 are re-sampled at a re-sampling frequency (2 Hz), which is set to the same value as the sampling frequency of the body motion signal of the body motion sensor 16, to set the sampling intervals to equal intervals. Specifically, the values on the interpolated dotted line 200 are re-sampled every 0.5 seconds as illustrated by point 211, point 212 . . . and point 223. As a result, a data string of equal internal RRIs is generated.

When the preprocessing in step S301 is ended, the feature value extractor 130 executes various types of feature value extraction processes for extracting each of the frequency-based feature values, the respiration-based feature values, and the time-based feature values described above (step S302).

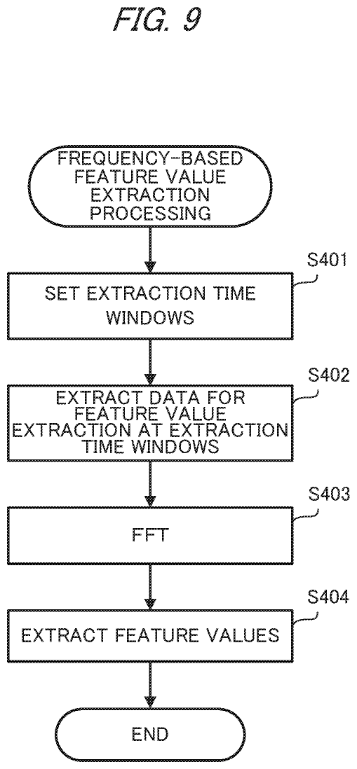

Of the various types of feature value extraction processes executed in step S302, FIG. 9 illustrates frequency-based feature value extraction processing for extracting the frequency-based feature values. First, an overview of the frequency-based feature value extraction processing will be described. In the frequency-based feature value extraction processing, frequency analysis is performed by FFT processing a data string rdata of equal interval RRIs extracted at various extraction time windows (described below), and a power spectrum distribution at frequency is calculated. A power spectrum of the low frequency band and a power spectrum of the high frequency band are obtained from the power spectrum distribution, and a plurality of parameters based on these power spectra are extracted as feature values.

The power spectrum of the low frequency band if (for example, from 0.01 Hz to 0.15 Hz) mainly represents the activity states of the sympathetic nerve and the parasympathetic nerve The power spectrum of the high frequency band hf (for example, from 0.15 Hz to 0.5 Hz) mainly represents the activity state of the parasympathetic nerve, and greater power spectra of the high frequency band hf are considered to indicate that the parasympathetic nerve of the subject is active.

The sympathetic nerve and the parasympathetic nerve have a set correlation with sleep states. The sleep states of a human can be classified into the stages of awake, REM sleep, and non-REM sleep. Non-REM sleep can be further classified into the stages of light sleep and deep sleep. During non-REM sleep, the sympathetic nerve is at rest and the heartbeat slows. In contrast, during REM sleep, the sympathetic nerve is as active as when awake, and the heartbeat is faster than during non-REM sleep. Accordingly, it is possible to estimate the sleep state using the power spectrum of the low frequency band if and the power spectrum of the high frequency band hf of the equal interval RRIs.

As illustrated in FIG. 9, in the frequency-based feature value extraction processing, first, extraction time windows for performing the FFT processing described above are set (step S401). The time window setter 120 sets the extraction time windows. The extraction time windows are periods for extracting the feature values from the RRIs and the acceleration norms acquired by the biological signal acquirer 110. In polysomnography (PSG), the sleep state is predicted in units of epochs, which are set amounts of time. In many cases, 30 seconds is the amount of time chosen for one epoch. Accordingly, the extraction time windows are set while shifting 30 seconds at a time on the time axis, and the feature values of the RRIs in the set extraction time windows are extracted. A variety of extraction time windows are set. Examples thereof include long time windows for extracting overall features, short time windows for extracting momentary features, and intermediate windows therebetween. Diverse feature values are acquired as a result of this configuration.

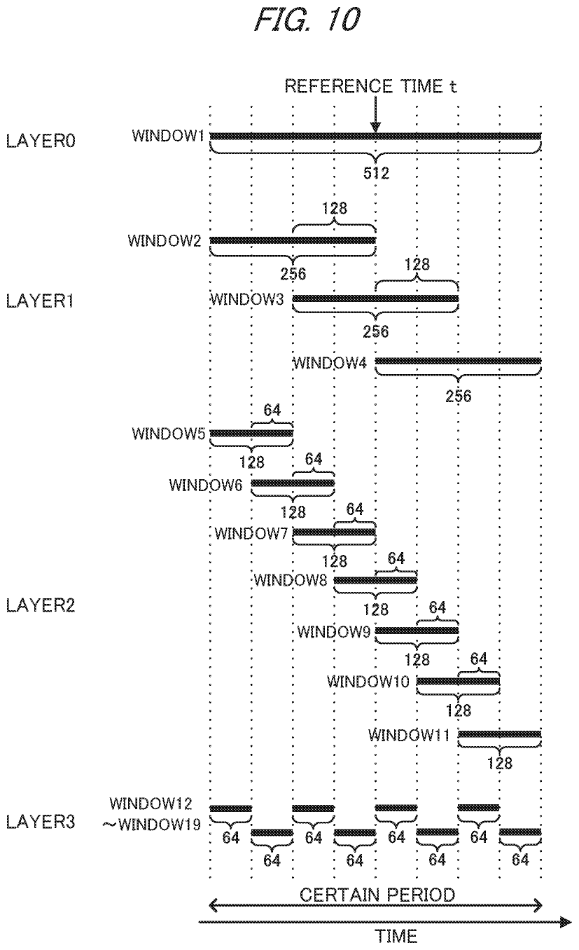

FIG. 10 illustrates extraction time windows set with respect to a reference time t of an epoch. In FIG. 10, time is represented on the horizontal axis. Extraction time windows having a plurality of mutually different periods are set from layer 0 to layer 3 in a period that is centered on the reference time t and that has a length of 256 sec (≈4 minutes) in which 512 sample points at a sampling frequency of 2 Hz are included. A window 1 that is a period having a time length of 256 seconds (≈4 minutes) is set in layer 0. The window 1 includes 512 (=2×256) sample points. Three extraction time windows, namely windows 2 to 4 that are periods having a time length of 128 seconds (≈2 minutes) are set in layer 1. The windows 2 to 4 are respectively set at mutually different positions on the time axis such that adjacent windows overlap 64 seconds (≈1 minute). Accordingly, the window 3 is provided centered on the reference point t. Each of the extraction time windows includes 256 sample points. Seven extraction time windows, namely windows 5 to 11 that are periods having a time length of 64 seconds (≈1 minute) are set in layer 2. The windows 5 to 11 are respectively set at mutually different positions on the time axis such that adjacent windows overlap 32 seconds. Accordingly, the window 8 is provided centered on the reference point t. Each of the extraction time windows includes 128 sample points. Eight extraction time windows, namely windows 12 to 19 that are periods having a time length of 32 seconds are set in layer 3. The windows 12 to 19 are set at mutually different positions on the time axis so as to be continuous without gaps. Each of the extraction time windows includes 64 sample points. Hereinafter, the data string extracted in each extraction time window from the data string of equal interval RRIs is expressed as rdata [p][i]. Note that p is the time window number, and i is the sample number. That is, p is a value from 1 to 19 and, if n is the number of samples in each extraction time window used in the extraction of the data string rdata [p][i], i is a value of 1 to n. The time length of each of the windows 1 to 19 is set to a value corresponding to the re-sampling frequency of the equal interval RRIs and the limit of the number of data in the FFT processing.

Data for the feature value extraction is extracted in accordance with the extraction time windows set in step S401 (step S402). The extraction time windows that are used differ depending on the feature values to be extracted. FFT processing is performed on the data that is extracted in accordance with the extraction time windows (step S403). In the frequency-based feature value extraction processing, the FFT processing is performed using all of the extraction time windows (windows 1 to 19) from layer 0 to layer 3. Upon performance of the FFT frequency analysis processing on the data string rdata [p][i] of the equal interval RRIs extracted in each extraction time window, and calculation of the frequency power vector density, extraction of the RRI feature values is performed for each extraction time window (step S404). In this case, in order to determine the sleep state, the following nine feature values that include the power spectrum of the low frequency band if and the power spectrum of the high frequency band hf described above are extracted as the RRI feature values.

Lf,hf,vlf,tf,hf_lfhf,lf_hf,vlf/tf,if/tf,hf/tf

Here,

lf: Power spectrum greater than 0.01 Hz and less than or equal to 0.15 Hz

hf: Power spectrum greater than 0.15 Hz and less than or equal to 0.5 Hz

vlf: Power spectrum less than or equal to 0.01 Hz

tf: vlf+lf+hf

hf_lfhf: hf/(hf+lf)

lf_hf: lf/hf

As described above, the number of windows in layer 0 is one, the number of windows in layer 1 is three, the number of windows in layer 2 is seven, and the number of windows in layer 3 is eight. Thus, a total of 19 extraction time windows are set. Since nine RRI feature values are extracted in each extraction time window, RRI feature values of a total of 171 (=9×19) dimensions are extracted.

Next, the respiration-based feature value extraction processing of the various feature value extraction processes executed in step S302 of FIG. 7 will be described. Respiration-based feature values are feature values related to respiration as the biological signal. The RRIs include a respiratory variation component, and variation of about 0.25 Hz occurs due to respiratory sinus arrhythmia (hereinafter referred to as “RSA”) caused by reduced heart rate due to expiration and increased heart rate due to inspiration. This RSA is used as a feature value related to respiration.

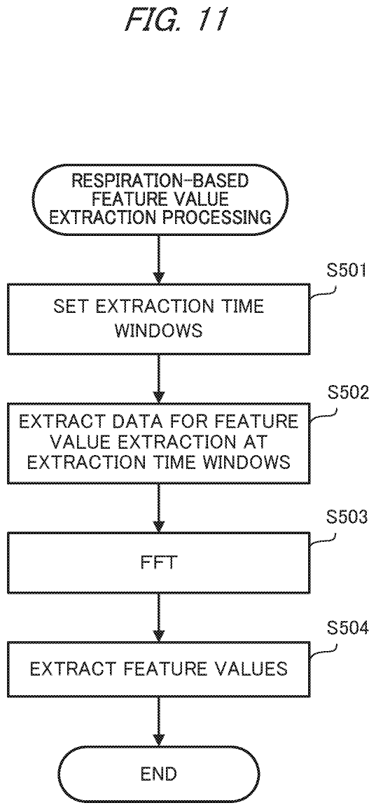

FIG. 11 illustrates the respiration-based feature value extraction processing for extracting the respiration-based feature values, executed in step S302 of FIG. 7. First, in step S501, the extraction time windows are set. Next, data for the feature value extraction is extracted in accordance with the extraction time windows set in step S501 (step S502). FFT processing is performed on the data extracted in accordance with the extraction time windows (step S503). In step S501 of the respiration-based feature value extraction processing, the windows 2 to 4 that are the three extraction time windows of layer 1 are set. Then, in steps S502 and S503, FFT frequency analysis processing is performed on the data strings of the equal interval RRIs extracted in each extraction time window (rdata[2][i], rdata[3][i], and rdata[4][i]), and the frequency in the range of 0.1 Hz to 0.4 Hz at which the power spectrum density is maximum is obtained as the RSA. When the RSA is obtained, extraction of the feature values related to respiration is performed for each extraction time window (step S504). Since respiration is stable in non-REM sleep states, the standard deviation is small. In contrast, in the awake state and in the REM sleep state, respiration is unstable and the standard deviation is large. Therefore, the following six feature values, which are thought to have correlation with sleep, among the respiration data included in the data strings of the equal interval RRIs are extracted.

For RSA[j] (where j corresponds to the extraction time window used in the extraction; (window 2: j=0, window 3: j=1, window 4; j=2)) obtained as described above:

mRSA: Average value of RSA[j]=(RSA[0]-PRSA[1]+RSA[2])/3
sdRSA: Standard deviation of RSA[j]=standard deviation obtained from RSA[0], RSA[1], and RSA[2]
minRSA: Minimum value of RSA[j]=min(RSA[0], RSA[1], RSA[2])
maxRSA: Maximum value of RSA[j]=max(RSA[0], RSA[1], RSA[2])
cvRSA: sdRSA/mRSA (where if mRSA 0, cvRSA=0) coefficient of respiratory variation RSA[1]: RSA of window 3 that is the center extraction time window

In the respiration-based feature value extraction processing a six-dimension feature values are extracted from the three extraction time windows, namely windows 2 to 4.

Next time-based feature value extraction processing of the various feature value extraction processes executed in step S302 of FIG. 7 will be described. The time-based feature value extraction processing includes RRI-based feature value extraction processing for extracting feature values obtained from the equal interval RRI data string rdata [p][i] (time series data) itself, and body motion-based feature value extraction processing for extracting feature values obtained from an acceleration data string mdata [p][i] itself. The acceleration data string mdata [p][i] is a data string of the acceleration norms calculated in step S205 of FIG. 5, and p and i are respectively the time window number and the sample number.

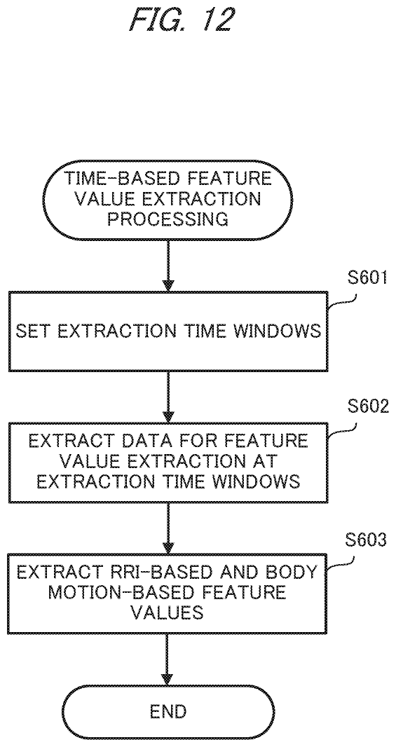

In the time-based feature value extraction processing, the extraction time windows are set in step S601 as illustrated in FIG. 12 and, thereafter, data for the feature value extraction is extracted in accordance with the set extraction time windows (step S602). In the time-based feature value extraction processing, all of the extraction time windows (windows 1 to 19) from layer 0 to layer 3 are set. Extraction of RRI-based feature values is performed on the RRI data string rdata [p][i] extracted in accordance with each extraction time window and, also, extraction of body motion-based feature values is performed on the acceleration data string mdata [p][i] (step S603). The RRI-based feature values and the body motion-based feature value are as follows.

RRI-Based Feature Values

The following six values, namely mRRI, mHR, sdRRI, cvRRI, RMSSD, and pNN50 are calculated (derived) for each extraction time window as RRI-based feature values. Note that the unit of values of rdata [p][i] is milliseconds, and the values are defined as follows:

mRRI[p]=Average value of rdata[p][i]

mHR[p]=60000/mRRI[p]

(Note: Since the heart rate per minute (mHR) is obtained from mRRI[p] which has milliseconds as the unit, 60 seconds×1000=60000 is divided by mRRI[p])

sdRRI[p]=Standard deviation of rdata[p][i]

cvRRI[p]=sdRRI[p]/mRRI[p]

RMSSD[p]=Square root of the average of the squares of the differences between mutually adjacent rdata[p][i]

pNN50[p]=Ratio of number of times the difference between mutually adjacent rdata[p][i] exceeds a certain amount of time (for example, 50 msec)

(Note: The ratio at which the difference between continuous adjacent RRIs exceeds 50 msec serves as an indicator of the degree of tension of the vagus nerve and, as such, 50 msec is used as the certain amount of time in the present embodiment)

The six values described above are obtained for each extraction time window and there are 19 extraction time windows. As such a total of 6×19=114 feature values are derived.

Body Motion-Based Feature Values

The magnitude and number of occurrences of body motions measured by the acceleration sensor differ for the awake state and for the sleep state. The following five values, namely mACT, sdACT, minACT, maxACT, and cvACT are derived for each extraction time window as body motion-based feature values. The various values are defined as follows:

mACT[p]: Average value of mdata[p][i]

sdACT[p]=Standard deviation of mdata[p][i]

minACT[p]=Minimum value of mdata[p][i]

maxACT[p]=Maximum value of mdata[p][i]

cvACT: sdACT[p]/mACT[p]

The five values described above are obtained for each extraction time window and there are 19 extraction time windows. As such a total of 5×19=95 feature values are derived.

In addition to the feature values described above, it is possible to extract, as a feature value, elapsed time from the occurrence of a large body motion to when a current estimation is performed in the periodically executed sleep state estimation. The body motion is expressed by the acceleration norm described above, and the average value mACT of the body motion is calculated for each reference point t. It is determined, based on mACT exceeding a certain threshold, that a large body motion has occurred. Four different values are set as the threshold. The elapsed time is measured by counting one each time a subsequent epoch reference point t is reached after a large body motion is detected. There are four count values, namely a count value from when mACT last exceeds 1.05, a count value from when mACT last exceeds 1.10, a count value from when mACT last exceeds 1.15, and a count value from when mACT last exceeds 1.20. Thus, four-dimension feature values are extracted. Here, mACT=1.0 indicates that the subject is still, and larger mACT values indicate larger body motions of the subject.

In FIG. 7, when the various feature values are calculated, feature value selection (dimension compression) processing for eliminating unnecessary feature values from the calculated feature values is executed (step S303). In one example, analysis of variance (ANOVA) or recursive feature elimination (RFE) is used as the feature value selection technique to eliminate the unnecessary feature values. In RPE, a mechanical learning model is actually used to learn and evaluate the feature set, confirm which features are important, and eliminate feature values until a designated number of features is met.

Upon elimination of the feature values by the dimension compression, feature value expansion processing for adding, as feature values, values obtained by moving the remaining feature values forward (future) or backward (past) on the time axis is executed (step S304). Note that the dimension compression processing (step S303) and the feature value expansion processing (step S304) are not required for the implementation of the present disclosure, and may be omitted.

The feature values subjected to the feature value expansion processing are smoothed in the time direction by a pre-filter (step S305). In one example, the pre-filter is a Gaussian filter. The pre-filter prevents the erroneous estimation of sleep states by not allowing data that unnaturally and suddenly changes to be input into the estimator 140. The extracted feature values are normalized between 0 and 1 and input into the estimator 140.

In FIG. 4, when the plurality of feature values (the frequency-based feature values, the respiration-based feature values, and the time-based feature values) is extracted by the feature value extractor 130, the estimator 140 estimates the sleep state based on the feature values extracted by the feature value extractor 130 (step S103). The sleep state estimation is performed in units of 30 seconds.

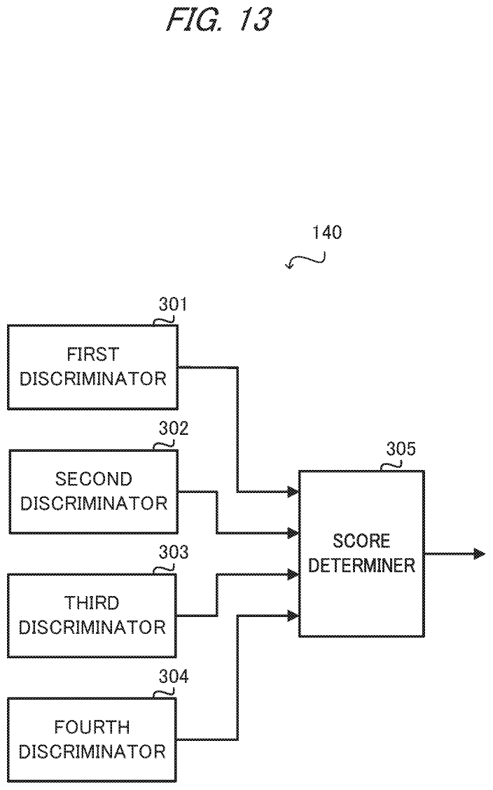

The estimator 140 is constituted from a multi-class discriminator obtained by combining two-class discriminators. In one example, multiclass-logistic regression is used for the discriminators. For example, it is possible to ascertain which combinations of explanatory variables (independent variables), namely the feature values such as tf, vlf, lf, hf, hf_lfhf, lf_hf, vlf/tf, lf/tf, and hf/tf significantly affect objective variables (dependent variables) divided into the two classes of [awake] indicating that the sleep state of the subject is the awake state and [other] indicating a state other than the awake state. Four outputs are set for the input feature values. Specifically, an output indicating that the sleep state is the awake state (awake), an output indicating that the sleep state is the REM sleep state (rem), an output indicating that the sleep state is the light sleep state (light), and an output indicating that the sleep state is the deep sleep state (deep) are set. As such, the estimator 140 is constituted from a combination of four two-class discriminators, as illustrated in FIG. 13. Specifically, the estimator 140 includes a first discriminator 301 that discriminates if the sleep state is the awake state [awake] or a state other than the awake state [other than awake], a second discriminator 302 that discriminates whether the sleep state is the REM sleep state [rem] or a state other than the REM sleep state [other than rem], a third discriminator 303 that discriminates whether the sleep state is the light sleep state [light] or a state other than the light sleep state [other than light], and a fourth discriminator 304 that discriminates whether the sleep state is the deep sleep state [deep] or a state other than the deep sleep state [other than deep]. Moreover, the estimator 140 includes a score determiner 305 that scores/determines the outputs of the first to fourth discriminators 301 to 304. Logistic regression expresses the objective variables and the explanatory variables by the following relational expression (sigmoid function).

f(x)=1/{1+exp[−(a1·x1+a2·x2+ . . . +an·xn+a0)]} Equation 1

Here, f(x) represents the occurrence probability of the event that is an objective variable, xn represents the various feature values that are the explanatory variables, an represents the learning coefficient (a parameter of a determination condition), and n represents the number of dimensions that is the number of types of data. The two classes are discriminated based on a size ratio of f(x) to 1-f(x). The relational expression described above clarifies the degree of influence of the explanatory variables, used in prediction value calculation and the relational expression, on the objective variables.

When the feature values are input into the first discriminator 301, the second discriminator 302, the third discriminator 303, and the fourth discriminator 304 that are one-to-other two-class discriminators, each of the discriminators performs discrimination processing.

The first discriminator 301 outputs a probability P1 (=f(x)) that the sleep state of the subject is the awake state, and compares the sizes of the probability P1 with a probability 1-P1 (=1-f(x)) that the sleep state is not the awake state. When, based on this comparison, the probability P1 is greater than the probability 1-P1, the probability P1 as the output indicates that the sleep state is the awake state and, when the probability 1-P1 is greater than the probability P1, the probability 1-P1 as the output indicates that the sleep state is a state other than the awake state. The second discriminator 302 outputs a probability P2 that the sleep state of the subject is the REM sleep state, and compares the sizes of the probability P2 with a probability 1-P2 that the sleep state is not the REM sleep state. When, based on this comparison, the probability P2 is greater than the probability 1-P2, the probability P2 as the output indicates that the sleep state is the REM sleep state and, when the probability 1-P2 is greater than the probability P2, the probability 1-P2 as the output indicates that the sleep state is a state other than the REM sleep state. The third discriminator 303 outputs a probability P3 that the sleep state of the subject is the light sleep state, and compares the sizes of the probability P3 with a probability 1-P3 that the sleep state is not the light sleep state. When, based on this comparison, the probability P3 is greater than the probability 1-P3, the probability P3 as the output indicates that the sleep state is the light sleep state and, when the probability 1-P3 is greater than the probability P3, the probability 1-P3 as the output indicates that the sleep state is a state other than the light sleep state. The fourth discriminator 304 outputs a probability P4 that the sleep state of the subject is the deep sleep state, and compares the sizes of the probability P4 with a probability 1-P4 that the sleep state is not the deep sleep state. When, based on this comparison, the probability P4 is greater than the probability 1-P4, the probability P4 as the output indicates that the sleep state is the deep sleep state and, when the probability 1-P4 is greater than the probability P4, the probability 1-P4 as the output indicates that the sleep state is a state other than the deep sleep state. A time direction Gaussian filter is provided for each of the class output results. The output datum are smoothed by the filters, thereby removing unnatural sudden output data variation and preventing erroneous sleep state estimation.

The outputs (the probabilities P1 to P4) of the first to fourth discriminators 301 to 304 that are smoothed by the Gaussian filters are input into the score determiner 305. The score determiner 305 compares the sizes of the outputs of the first to fourth discriminators 301 to 304, estimates that the sleep state of the subject is the sleep state that corresponds to the maximum value of the probabilities P1 to P4, and outputs data expressing that sleep state to the user interface 13. Thus, the estimated sleep state is presented to the user.

The parameter an of the determination condition is determined by machine learning using sample data. Since the sleep state estimation by the estimator 140 is performed in units of 30 seconds, a label, on which sleep states estimated by an expert every 30 seconds based on an EEG, an ECG, or the like are recorded, is added to the sample data of the feature values.

The sample data is prepared for each of the awake state, the REM sleep state, the light sleep state, and the deep sleep state. The feature value extraction processing is performed for the prepared sample data. The data of the extracted feature values and the label are associated and stored as teacher data. Note that, the machine learning may be performed in the discrimination device described above, or may be performed in another device and a determination model may be stored in the discrimination device.

In the aforementioned description, the estimator 140 is formed by using a sigmoid function as the activation function and providing a plurality of one-to-other two-class discriminators. However, the estimator 140 is not limited thereto, and a softmax function may be used as the activation function to form the multi-class discriminator. Additionally, the estimator 140 may be formed by providing a plurality of one-to-one two-class discriminators. Furthermore, in the aforementioned description, logistic regression is used as the machine learning algorithm, but the machine learning algorithm is not limited thereto and a support vector machine, a neural network, a decision tree, a random forest, or the like may be used. Moreover, a plurality of algorithms may be combined and used. Examples thereof include support vector classification (SVC), support vector regression (SVR), support vector classification (SVC), and eXtreme gradient boosting (xgboost).

In FIG. 4, the sleep state estimation by the estimator 140 is carried out in real-time in units of 30 seconds. The controller 11 determines whether an end condition is satisfied (step S104). Examples of the end condition include when the elapsed time from the start of processing reaches a set time, when there is an end instruction from the input device, and the like. When the end condition is not satisfied (step S104: NO), step S101 is returned to and the sleep state estimation processing is continued. When the end condition is satisfied (step S104: YES), the sleep state prediction processing is ended.

Thus, according to the present embodiment, a plurality of extraction time windows having mutually different time lengths are set in the certain period in which the biological signal is being acquired, and the feature values of the biological signal in each of the plurality of set extraction time windows are extracted. As a result, it is possible to extract, as the feature values, macro features whereby the biological signal changes over a long period of time, and micro features whereby the biological signal changes over a short period of time. For example, in a case in which the features whereby the biological signal changes over time are a major factor in estimating the sleep state, if the extraction time windows for the feature value extraction are short, it is not possible to extract the feature values that change over time. In this case, by estimating based on the feature values extracted using the window 1 that extends greatly into the past and the future in the certain period, it is possible to correctly estimate the sleep state based on the thusly estimated feature values.

In contrast, in a case in which local, short-time changes of the biological signal are feature values that are a major factor in determining the sleep state, if the time lengths of the extraction time windows for the feature value extraction are long, it is not possible to ascertain the local changes as feature values. In this case, by shortening the time lengths of the extraction time windows for the feature value extraction, local changes can be extracted as feature values, and it is possible to correctly estimate the sleep state based on the thusly estimated feature values. Additionally, in a determination of the sleep state based on the feature values of the biological signal in the certain period, cases are possible in which a local change of the biological signal at a point somewhat in the past is a major determination factor of the sleep state. Furthermore, this local change of the biological signal may occur at a plurality of mutually different points in time. In such a case, it is possible to improve the accuracy of the sleep state estimation by shortening the time lengths of the extraction time windows for the feature value extraction and setting, in a certain period on the time axis, the extraction time windows at a plurality of positions in the past. Additionally, the feature values can be extracted thoroughly (that is, without omission) by partially overlapping, on the time axis, extraction time windows that are adjacent to each other on the time axis. Moreover, the feature values can be extracted without omission from the biological signal acquired in the certain period by setting the extraction time windows in the certain period so as to be continuous. The positions and ranges of the occurrences of the feature values that are major factors in the determination of the sleep state are thought to be diverse. Accordingly, by setting extraction time windows having different time lengths at mutually different positions on the time axis, and using the feature values obtained from the extraction time windows together, it is possible to capture macro changes and micro changes of the feature values without omission, and improve the accuracy of the sleep state estimation.

Furthermore, since four sets of time windows (the time windows of layers 0 to 3), which is more than two sets, are set as the plurality of extraction time windows, it is possible to compensate for the influence of the uncertainty principle and further improve the accuracy of the sleep state estimation.

Furthermore, the feature values to be extracted may be changed depending on the purpose of the sleep state estimation. For example, in a case in which the overall sleep state tendencies while sleeping are to be ascertained, feature values obtained from extraction time windows having long time lengths may be selected and, in a case in which the details of the sleep state at a certain point in time while sleeping are to be ascertained, feature values obtained from extraction time windows having short time lengths may be selected.

As a result of the sleep state estimation processing described above, the state estimation apparatus 10 can estimate, in real-time, the sleep state even if the subject is sleeping.

The sleep states of 45 subjects were estimated according to the present embodiment. The total_accuracy and the total_kappa that represent estimation accuracy were respectively 0.7229 and 0.5960. In contrast, the feature values of a biological signal were extracted as in the related art described above using a single extraction time window (30 seconds), and the sleep states of the same 45 subjects were estimated based on the extracted feature values of the biological signal. In this case, the total_accuracy was 0.6942 and the total_kappa was 0.5576. Thus, it was found that the sleep states of subjects can be more accurately estimated using the present embodiment than when a single extraction time window is used.

In the embodiment described above, the number and/or the time lengths of extraction time windows may be variably set according to various conditions and parameters. In one example, the number and/or the time lengths of extraction time windows changes according to changes in the sleep state of the subject. In this case, in order to improve the accuracy of sleep state estimation in a situation in which the sleep state changes, the extraction time windows are set so as to extract more feature values. In contrast, in a situation in which the sleep state does not change, the calculation load is reduced by reducing the number of extraction time windows and setting longer time lengths for the extraction time windows. Situations in which the sleep state changes are determined, for example, based on a degree of change of the outside atmosphere around the subject, a degree of change of illuminance around the subject, a degree of change of the core body temperature of the subject and the like (at least one of these). The CPU functions as a determination device to perform this determination. The core body temperature of the subject can be detected from an ear or the head of the subject by using a sensor or the like. Additionally, situations in which the sleep state changes can be determined based on the heart rate or body motion or the like, or based on the feature values themselves. Alternatively, the number and/or the time lengths of the extraction time windows may be set so as to be mutually different for cases when the subject (human) is at rest and when the subject is active. To determine the state of the subject, the CPU functions as a determination device to perform the determination of whether the subject is at rest or active. This determination is performed based on the detection results of the body motion sensor 16 or the like.

Furthermore, the number and/or the time lengths of the extraction time windows may be set according to individual information about the subject, including the gender and/or the age of the subject. In this case, the individual information about the subject (human) is input by the user via the user interface 13, and the CPU functions as an individual information acquisition device to acquire the input individual information.

Additionally, in the embodiment described above, the plurality of extraction time windows having relatively short time lengths (windows 12 to 19) are set so as to be mutually continuous on the time axis without overlapping each other or having gaps between each other. However, this plurality of extraction time windows may be set such that gaps exist between the extraction time windows on the time axis. In such a case, it is possible to reduce the amount of data of the feature values while suppressing decreases in the accuracy of the feature values of the biological signal.

In the embodiment described above, the pulse wave sensor 15 for detecting the pulse wave of the subject and the body motion sensor 16 for detecting the movement of the body of the subject are provided. However, the types of sensors are not limited thereto. Any type of sensor that acquires a biological signal of the subject can be provided. Additionally, in a case in which the biological signal or the feature values of the biological signal can be received from an external device or the like via the communicator 14, for example, the state estimation apparatus 10 need not include a sensor.

In the embodiment described above, the state estimation apparatus 10 uses, as the biological signal, a pulse wave obtained by a pulse wave sensor worn on the earlobe and acceleration obtained by an acceleration sensor worn on the earlobe. However, the biological signal is not limited thereto. Examples of biological signals usable by the state estimation apparatus 10 include body motion (detected by an acceleration sensor provided on the head, arm, chest, foot, torso, or the like), EMG (detected by myoelectric sensors attached to the head (around the temples and nose), arms, chest, feet, torso, or the like), perspiration (detected by a skin electrometer or a humidity sensor), heartbeat (detection by an electrocardiograph, a pressure sensor installed under a bed (detection of ballistocardiogram waveform), a pulse wave sensor installed on the head, arm, chest, feet, torso, or the like), and the like.

In the embodiment described above, the state estimation apparatus 10 uses the frequency-based feature values based on the RRIs, the time-based feature values based on the RRIs and the body motion, and the respiration-based feature value as the feature values for estimating the sleep state. However, the feature values are not limited thereto and any desired type of feature values can be used provided that the feature values are feature values for estimating the sleep state. Moreover, the number of feature values is not limited. For example, in a case in which EMG, perspiration, or the like is used as the biological signal, as with the other feature values, the detection values thereof may be sampled at 2 Hz for example, and data may be extracted in a plurality of extraction time windows centered on the reference point t to calculate the feature values.

In the embodiment described above, the state estimation apparatus 10 estimates the sleep state of a human subject. However, the subject for which the sleep state is to be estimated is not limited to humans, and it is possible to set a dog, a cat, a horse, a cow, a pig, a chicken, or the like as the subject. These subjects can be used because it is possible to affix a pulse wave sensor and an acceleration sensor to these subjects and acquire feature values needed to estimate the sleep state. Additionally, in the embodiment described above, the sleep state is estimated as the state of the subject. However, instead of the sleep state, an emotion (joy, sadness, anger, resignation, surprise, disgust, fear, relaxation, or the like) may be estimated. For example, the sympathetic nerve actively works when in a tense state such as when under stress. In contrast, the parasympathetic nerve actively works when in a relaxed state. The sympathetic nerve works to accelerate the heartbeat, and the parasympathetic nerve works to slow the heartbeat. Accordingly, it is possible to estimate that the subject is in a relaxed state using feature values based on the power spectra of the low frequency band if and the high frequency band hf of the RRIs. Additionally, it goes without saying that feature values of other appropriate biological signals suited for estimating states of the subject may be extracted.

In the embodiment described above, the controller 11 functions as the biological signal acquirer 110, the time window setter 120, the feature value extractor 130, and the estimator 140 by the CPU executing the program stored in the ROM. However, the controller 11 may include, for example, an Application Specific Integrated Circuit (ASIC), a Field-Programmable Gate Array (FPGA), various control circuitry, or other dedicated hardware, and this dedicated hardware may function as the biological signal acquirer 110, the time window setter 120, the feature value extractor 130, and the estimator 140. In this case, the functions of each of the components may be realized by individual pieces of hardware, or the functions of each of the components may be collectively realized by a single piece of hardware. Additionally, the functions of each of the components may be realized in part by dedicated hardware and in part by software or firmware.

It is possible to provide a state estimation apparatus device that is provided in advance with the configurations for realizing the functions according to the present disclosure, and it is also possible to apply a program to cause an existing information processing device or the like to function as the state estimation apparatus according to the present disclosure. Any method may be used to apply the program. For example, the program can be applied by storing the program on a non-transitory computer-readable recording medium such as a flexible disc, a compact disc (CD) ROM, a digital versatile disc (DVD) ROM, and a memory card. Furthermore, the program can be superimposed on a carrier wave and applied via a communication medium such as the internet. For example, the program may be posted to and distributed via a bulletin board system (BBS) on a communication network. Moreover, a configuration is possible in which the processing described above is executed by starting the program and, under the control of the operating system (OS), executing the program in the same manner as other applications/programs.

The foregoing describes some example embodiments for explanatory purposes. Although the foregoing discussion has presented specific embodiments, persons skilled in the art will recognize that changes may be made in form and detail without departing from the broader spirit and scope of the invention. Accordingly, the specification and drawings are to be regarded in an illustrative rather than a restrictive sense. This detailed description, therefore, is not to be taken in a limiting sense, and the scope of the invention is defined only by the included claims, along with the full range of equivalents to which such claims are entitled.

What is claimed is:

1. A state estimation apparatus comprising:
at least one processor; and
a memory configured to store a program executable by the at least one processor;
wherein
the at least one processor is configured to:
acquire a biological signal of a subject,
in a certain period in which the biological signal is being acquired, set as a plurality of extraction time windows a plurality of time windows having mutually different time lengths,
extract a feature value of the biological signal in each of the plurality time windows, and
estimate a state of the subject based on the extracted feature value.

2. The state estimation apparatus according to claim 1, wherein the at least one processor is configured to further set, as the plurality of extraction time windows, a plurality of time windows having identical time lengths at mutually different positions on a time axis.

3. The state estimation apparatus according to claim 1, wherein at least two of the plurality of extraction time windows are set so as to have a gap therebetween on a time axis.

4. The state estimation apparatus according to claim 1, wherein at least two of the plurality of extraction time windows are set so as to partially overlap each other on a time axis.

5. The state estimation apparatus according to claim 1, wherein at least two of the plurality of extraction time windows are set so as to be continuous with each other without overlapping and without a gap therebetween on a time axis.

6. The state estimation apparatus according to claim 1, wherein the biological signal of the subject is at least one selected from a pulse wave and a body motion.

7. The state estimation apparatus according to claim 6, wherein the feature value is at least one selected from the group consisting of a frequency-based feature value based on a frequency of a heartbeat interval obtained from the pulse wave of a human that is the subject, a time-based feature value obtained from time series data of the heartbeat interval, a time-based feature value obtained from time series data of the body motion of the human, and a respiration-based feature value that is a feature value based on a respiratory variation component included in the heartbeat interval.

8. The state estimation apparatus according to claim 7, wherein the respiration-based feature value is at least one selected from the group consisting of an average value of the respiratory variation component in the plurality of extraction time windows, a standard deviation of the respiratory variation component in the plurality of extraction time windows, a minimum value of the respiratory variation component in the plurality of extraction time windows, and a maximum value of the respiratory variation component in the plurality of extraction time windows.

9. A state estimation method comprising:
acquiring a biological signal of a subject;
in a certain period in which the biological signal is being acquired, setting as a plurality of extraction time windows a plurality of time windows having different time lengths;
extracting a feature value of the biological signal in each of the plurality of extraction time windows; and
estimating a state of the subject based on the extracted feature value.

10. A non-transitory recording medium storing a program that causes a computer to:
acquire a biological signal of a subject;
in a certain period in which the biological signal is being acquired, set as a plurality of extraction time windows a plurality of time windows having different time lengths;
extract a feature value of the biological signal in each of the plurality of extraction time windows; and
estimate a state of the subject based on the extracted feature value.
